(19) United States
(12) Patent Application Publication
(10) Pub. No.: US 2021/0173104 A1
(43) Pub. Date: Jun. 10, 2021

(54) RADIATION DETECTOR WITH SUBPIXELS OPERATING IN DIFFERENT MODES

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 17/178,755

(22) Filed: Feb. 18, 2021

Related U.S. Application Data
(63) Continuation of application No. PCT/CN2018/104594, filed on Sep. 7, 2018.

(51) Int. Cl. G01T 1/29; G01V 5/00

(52) U.S. Cl. CPC G01T 1/2985; A61B 6/14; G01V 5/0025; G01V 5/005

(57) ABSTRACT

Disclosed herein is a radiation detector, comprising a pixel comprising a plurality of subpixels. Each of the subpixels configured to generate an electrical signal upon exposure to a radiation. The detector further comprises a switch electrically connected to the plurality of subpixels. The switch is configured to combine electrical signals generated by a subset of the subpixels. Disclosed also herein is a method in relation to the radiation detector.

### TECHNICAL FIELD

The disclosure herein relates to a radiation detector, in particular to a radiation detector having subpixels operating in different modes.

### BACKGROUND

A radiation detector is a device that measures a property of a radiation. Examples of the property may include a spatial distribution of the intensity, phase, and polarization of the radiation. The radiation may be one that has interacted with a subject. For example, the radiation measured by the radiation detector may be a radiation that has penetrated or reflected from the subject. The radiation may be an electromagnetic radiation such as infrared light, visible light, ultraviolet light, X-ray or γ-ray. The radiation may be of other types such as α-rays and β-rays.

One type of radiation detectors is based on interaction between the radiation and a semiconductor. For example, a radiation detector of this type may have a semiconductor layer that absorbs the radiation and generate charge carriers (e.g., electrons and holes) and circuitry for detecting the charge carriers.

### SUMMARY

Disclosed herein is a radiation detector, comprising: a pixel comprising a plurality of subpixels, each of the subpixels configured to generate an electrical signal upon exposure to a radiation; a switch electrically connected to the plurality of subpixels; wherein the switch is configured to combine electrical signals generated by a subset of the subpixels.

According to an embodiment, the switch is configured to detect a magnitude of the electrical signal generated by each of the subpixels.

According to an embodiment, the switch is configured to disconnect any one of the subpixels when the magnitude of the electrical signal generated by that subpixel exceeds a magnitude threshold.

According to an embodiment, the switch comprises a plurality of sub-switches respectively connected to the subpixels.

According to an embodiment, each of the subpixels is configured to detect a magnitude of the electrical signal generated by the subpixel connected thereto.

According to an embodiment, each of the sub-switches is configured to disconnect the subpixel connected thereto when the magnitude exceeds a magnitude threshold.

According to an embodiment, the pixel comprises four subpixels.

According to an embodiment, the pixel further comprises a radiation absorption layer.

According to an embodiment, the radiation absorption layer comprises a semiconductor.

According to an embodiment, the semiconductor is selected from a group consisting of silicon, germanium, GaAs, CdTe, CdZnTe, or combinations thereof.

According to an embodiment, the switch further comprises an accumulator to combine the electrical signals generated by any subset of the subpixels.

According to an embodiment, the radiation detector further comprises a comparator configured to compare an output signal from the switch to an output threshold; a counter configured to register a number of particles of radiation absorbed by radiation detector; a controller; a meter configured to measure the output signal; wherein the controller is configured to start a time delay from a time at which the comparator determines that an absolute value of the output signal equals or exceeds an absolute value of the output threshold; wherein the controller is configured to cause the meter to measure the output signal upon expiration of the time delay; wherein the controller is configured to determine a number of particles by dividing the output signal measured by the meter by an output signal of the switch that a single particle generates; wherein the controller is configured to cause the number registered by the counter to increase by the number of particles.

According to an embodiment, the controller is configured to deactivate the comparator at a beginning of the time delay.

Disclosed herein is a system comprising any of the radiation detectors above and an X-ray source, wherein the system is configured to perform X-ray radiography on human chest or abdomen.

Disclosed herein is a system comprising any of the radiation detectors above and an X-ray source, wherein the system is configured to perform X-ray radiography on human mouth.

Disclosed herein is a cargo scanning or non-intrusive inspection (NII) system comprising any of the radiation detectors above and an X-ray source, wherein the cargo scanning or non-intrusive inspection (NII) system is configured to form an image using backscattered X-ray.

Disclosed herein is a cargo scanning or non-intrusive inspection (NII) system comprising any of the radiation detectors above and an X-ray source, wherein the cargo scanning or non-intrusive inspection (NII) system is configured to form an image using X-ray transmitted through an object inspected.

Disclosed herein is a full-body scanner system comprising any of the radiation detectors above and an X-ray source.

Disclosed herein is an X-ray computed tomography (X-ray CT) system comprising any of the radiation detectors and an X-ray source.

Disclosed herein is an electron microscope comprising any of the radiation detectors, an electron source and an electronic optical system.

Disclosed herein is a system comprising any of the radiation detectors above, wherein the system is an X-ray telescope, or an X-ray microscopy, or wherein the system is configured to perform mammography, industrial defect detection, microradiography, casting inspection, weld inspection, or digital subtraction angiography.

Disclosed herein is a method comprising: obtaining a radiation detector comprising a pixel, wherein the pixel comprises a plurality of subpixels, each of the subpixels being configured to generate an electrical signal upon exposure to a radiation; identifying a subset of the subpixels; combining the electrical signals generated by the subset of the subpixels.

According to an embodiment, in the above-mentioned method, the radiation detector comprises a switch electrically connected to the plurality of subpixels, and the switch comprises a plurality of sub-switches respectively connected to the subpixels.

According to an embodiment, the method further comprises detecting a magnitude of the electrical signal generated by each subpixel using the sub-switch connected thereto.

According to an embodiment, the method further comprising disconnecting the subpixel using the sub-switch connected thereto upon determination that the magnitude exceeds a magnitude threshold.

### BRIEF DESCRIPTION OF FIGURES

FIG. 1 schematically shows a radiation detector, according to an embodiment.

FIG. 2 schematically shows a pixel of the radiation detector in FIG. 1, wherein the pixel comprises a plurality of subpixels.

FIG. 3 schematically shows a cross-sectional view of the radiation detector.

FIG. 4A schematically shows a detailed cross-sectional view of the radiation detector.

FIG. 4B schematically shows an alternative detailed cross-sectional view of the radiation detector.

FIG. 5 schematically shows a component diagram of a switch of the radiation detector in FIG. 4A or 4B, according to an embodiment.

FIG. 6 schematically shows a component diagram of an electronic system of the radiation detector in FIG. 4A or FIG. 4B, according to an embodiment.

FIG. 7 shows a temporal change of the output signal of the switch in FIG. 5 or FIG. 6, caused by charge carriers generated by one or more particles incident on the diode or the resistor, according to an embodiment.

FIG. 8 schematically shows a flow chart for a method suitable for using a radiation detector according to an embodiment.

FIG. 9 shows a flow chart for a method suitable for detecting radiation using a system such as the system operating as shown in FIG. 4.

FIG. 10 schematically shows a system comprising the radiation detector described herein, suitable for medical imaging such as chest X-ray radiography, abdominal X-ray radiography, etc., according to an embodiment.

FIG. 11 schematically shows a system comprising the radiation detector described herein suitable for dental X-ray radiography, according to an embodiment.

FIG. 12 schematically shows a cargo scanning or non-intrusive inspection (NII) system comprising the radiation detector described herein, according to an embodiment.

FIG. 13 schematically shows another cargo scanning or non-intrusive inspection (NII) system comprising the radiation detector described herein, according to an embodiment.

FIG. 14 schematically shows a full-body scanner system comprising the radiation detector described herein, according to an embodiment.

FIG. 15 schematically shows an X-ray computed tomography (X-ray CT) system comprising the radiation detector described herein, according to an embodiment.

FIG. 16 schematically shows an electron microscope comprising the radiation detector described herein, according to an embodiment.

### DETAILED DESCRIPTION

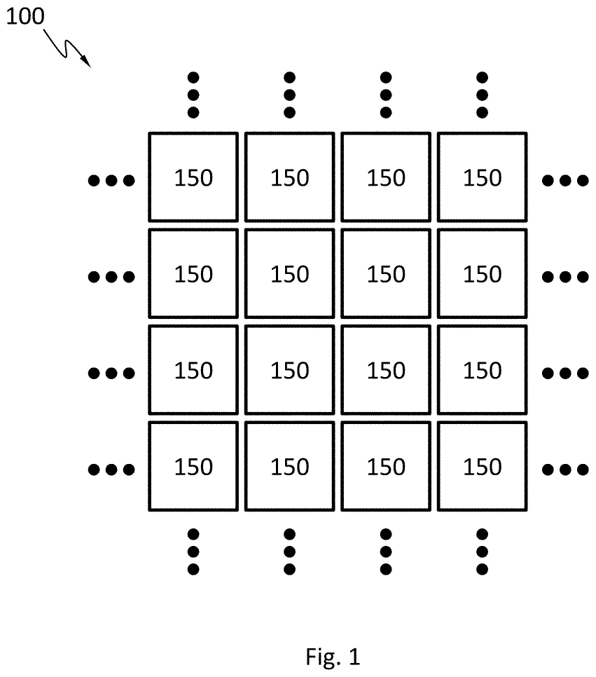

FIG. 1 schematically shows a radiation detector 100, as an example. The radiation detector 100 has an array of pixels 150. The array may be a rectangular array, a honeycomb array, a hexagonal array or any other suitable array. Each pixel 150 is configured to detect radiation from a radiation source incident thereon and may be configured measure a characteristic (e.g., the energy of the particles, the wavelength, and the frequency) of the radiation.

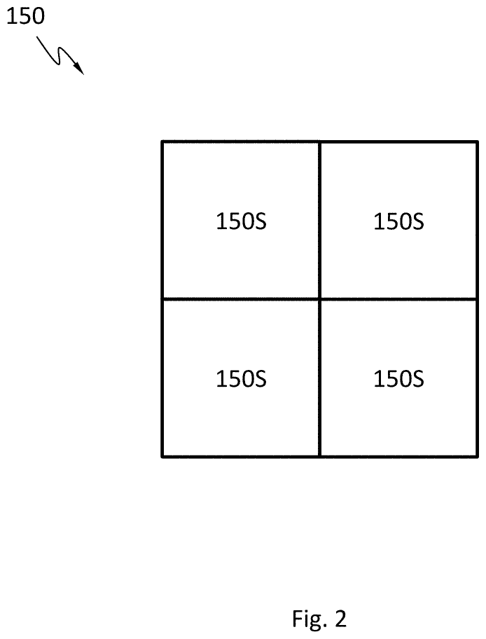

FIG. 2A schematically shows that a pixel 150 may include a plurality of subpixels 150S. In the example shown, the pixel 150 includes four subpixels 150S. However, the pixel 150 may include any suitable number of subpixels 150S. The subpixels 150S may each be configured to generate an electrical signal upon exposure to a radiation. The characteristic measured by the pixel 150 may be determined based on the electrical signals from the subpixels 150S included in the pixel 150. For example, the subpixels 150S may each be configured to count a number of particles of radiation incident thereon that have energies within a particular bin, within a period of time. The number of the particles of radiation incident on the pixel 150 that have energies within that particular bin within that period of time can be determined by adding the numbers counted by the subpixels 150S for that bin within that period of time. When the incident particles of radiation have similar energy, the subpixels 150S may each be configured to simply count a number of particles of radiation incident thereon within a period of time, without measuring the energy of the particles of radiation. The number of the particles of radiation incident on the pixel 150 within that period of time can be determined by adding the numbers counted by the subpixels 150S within that period of time.

Each of the subpixels 150S may have its own analog-to-digital converter (ADC) configured to digitize the electrical signal it generates. The subpixels 150S may be configured to operate in parallel, and operate independently from one another. For example, malfunction of one subpixel 150S would not affect the normal operation of another subpixel 150S in the same pixel 150. For example, when one subpixel 150S measures a particle of radiation, another subpixel 150S may be waiting for a particle of radiation to arrive. The subpixels 150S may or may not be individually addressable.

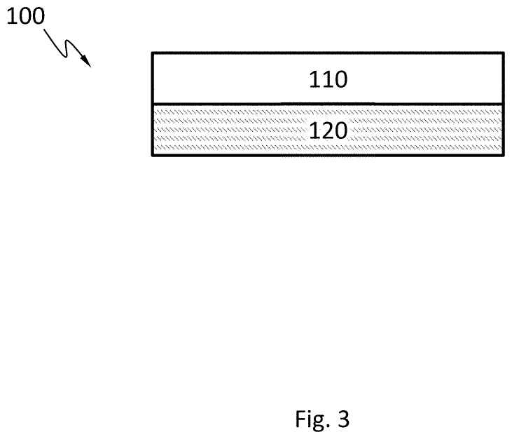

FIG. 3 schematically shows a cross-sectional view of the radiation detector 100, according to an embodiment. The radiation detector 100 may include a radiation absorption layer 110 and an electronics layer 120 (e.g., an ASIC) for processing or analyzing electrical signals incident radiation generates in the radiation absorption layer 110. Each of the pixels 150 may include a portion of the radiation absorption layer 110. The radiation detector 100 may or may not include a scintillator. The radiation absorption layer 110 may include a semiconductor material such as, silicon, germanium, GaAs, CdTe, CdZnTe, or a combination thereof. The semiconductor may have a high mass attenuation coefficient for the radiation of interest.

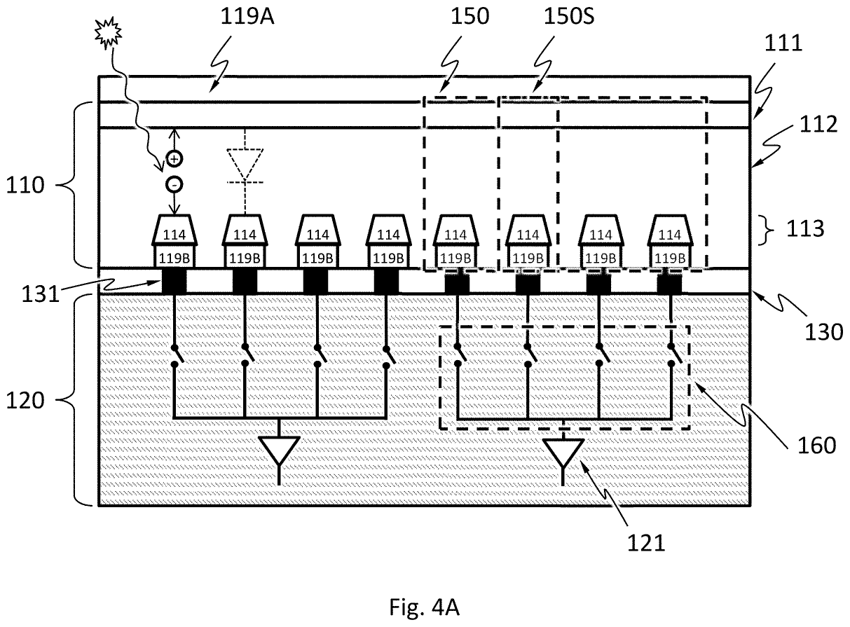

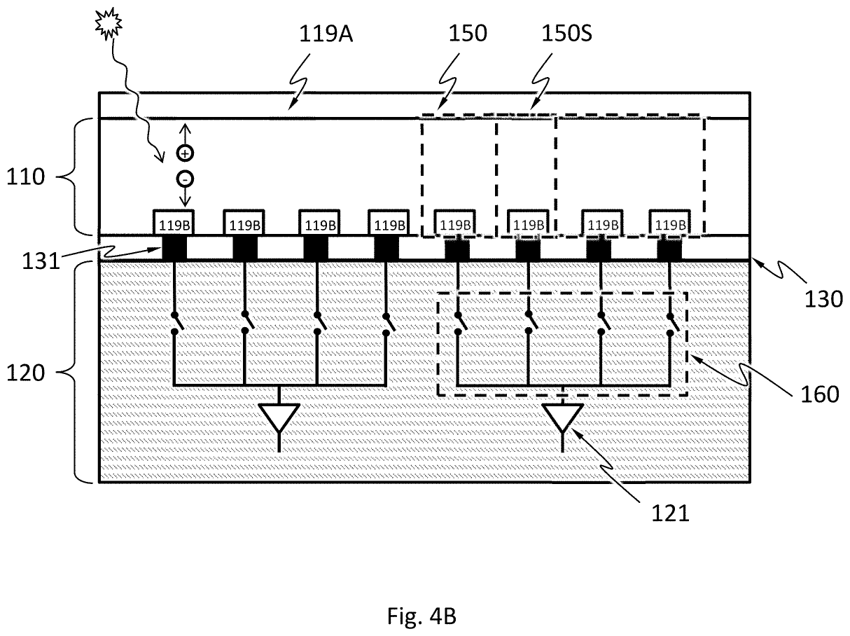

As shown in a detailed cross-sectional view of the radiation detector 100 in FIG. 4A, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. In an embodiment, the discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 4A, each of the discrete regions 114 of the second doped region 113 forms a diode with the first doped region 111 and the optional intrinsic region 112. Namely, in the example in FIG. 4A, t he radiation absorption layer 110 has a plurality of diodes having the first doped region 111 as a shared electrode. The first doped region 111 may also have discrete portions. A subpixel 150S may encompass one of the discrete regions 114. A pixel 150 may encompass a plurality of adjacent subpixels 150S.

When a particle of radiation from the radiation source hits the radiation absorption layer 110 including diodes, the particle of radiation may be absorbed and generate one or more charge carriers by a number of mechanisms. The charge carriers may drift to the electrodes of one of the diodes under an electric field. The field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A subpixel 150S associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the subpixel.

As further shown in FIG. 4A, the subpixels 150S of the pixel 150 are electrically connected to a switch 160. The switch 160 is configured to combine the electrical signals generated by any subset of the subpixels 150S of the pixel 150. In the present disclosure, the subset always has fewer subpixels 150S than the total number of subpixels 150S in the pixel 150. For example, if the pixel 150 has four subpixels 150S, the subset may have three subpixels 150S, two subpixels 150S, one subpixel 150S, or zero subpixel 150S. In an embodiment, the magnitude of the electrical signal generated by every subpixel 150S in the subset is below a magnitude threshold. In an embodiment, the magnitude of the electrical signal generated by every subpixel 150S not in the subset is above the magnitude threshold. In an embodiment, the magnitude threshold is an upper limit of the magnitude of the electrical signal a non-defective subpixel 150S generates when not receiving a particle of radiation. Namely, the magnitude threshold may be an upper limit of the dark current in a non-defective subpixel 150S. In other words, the subset may consist of all the non-defective subpixels 150S of the pixel 150.

In an embodiment, the switch 160 is configured to detect the magnitude of the electrical signal generated by each of the subpixels 150S. The switch 160 may disconnect a subpixel 150S, when it has detected that the magnitude of the subpixel 150S exceeds the magnitude threshold. Namely, the switch 160 may exclude any of the subpixels 150S from the subset based on the magnitude of the electrical signal it generates. In an embodiment, the disconnected subpixel 150S is grounded.

As shown in an alternative detailed cross-sectional view of the radiation detector 100 in FIG. 4B, according to an embodiment, the radiation absorption layer 110 may include a resistor of a semiconductor material such as, silicon, germanium, GaAs, CdTe, CdZnTe, or a combination thereof, but does not include a diode. The semiconductor may have a high mass attenuation coefficient for the radiation of interest.

When a particle of radiation hits the radiation absorption layer 110 including a resistor but not diodes, it may be absorbed and generate one or more charge carriers by a number of mechanisms. A particle of the radiation may generate 10 to 100000 charge carriers. The charge carriers may drift to the electrical contacts 119A and 119B under an electric field. The field may be an external electric field. The electrical contact 119B includes discrete portions. A subpixel 150S may encompass one of the discrete portions. A pixel 150 may encompass a plurality of adjacent subpixels 150S. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A subpixel 150S associated with a discrete portion of the electrical contact 119B may be an area around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the subpixel 150S associated with the one discrete portion of the electrical contact 119B.

In the embodiment as shown in FIG. 4B, the subpixels 150S of the pixel 150 are electrically connected to a switch 160. The switch 160 is configured to combine the electrical signals generated by any subset of the subpixels 150S of the pixel 150, in a manner as similarly mentioned above in connection with FIG. 4A.

Similarly, the switch 160 is configured to detect a magnitude of the electrical signal generated by each of the subpixels 150S. The switch 160 further disconnects a subpixel 150S, when it has detected that the magnitude of the subpixel 150S equals to or exceeds a magnitude threshold, in a similar manner as mentioned above in connection with FIG. 4A.

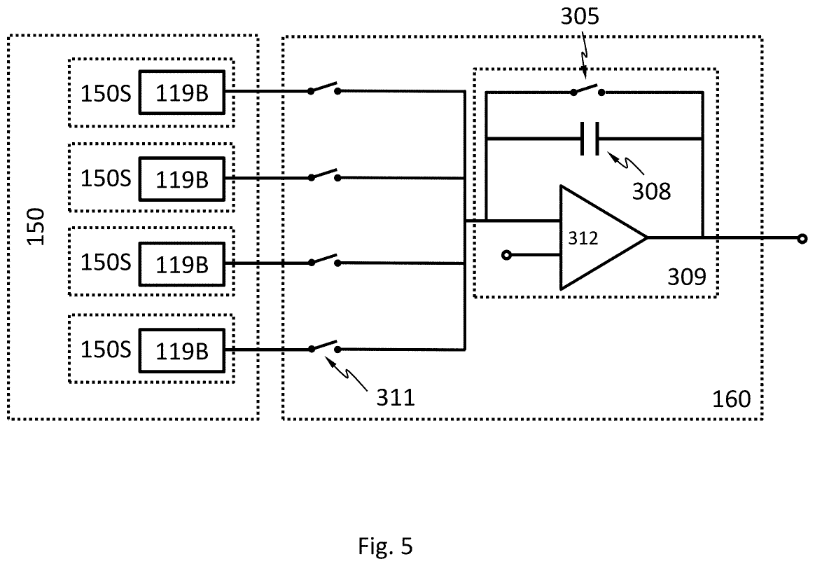

FIG. 5 schematically shows a component diagram of the switch 160, according to an embodiment. The switch 160 may comprise a plurality of sub-switches 311 respectively connected to the plurality of subpixels 150S of a pixel 150. In an embodiment as shown in FIG. 5, the sub-switches 311 are respectively connected to discrete portions of the electrical contact 119B associated with the subpixels 150S. Each of the sub-switches 311 is configured to detect the magnitude of the electrical signal generated by the subpixel 150S connected thereto, and configured to disconnect the subpixel 150S when it detects that the magnitude exceeds the magnitude threshold. Namely, the sub-switches 311 may exclude any of the subpixels 150S from the subset based on the magnitude of the electrical signal it generates. In an embodiment, the disconnected subpixel 150S is grounded.

In an embodiment, the switch 160 is configured to combine the electrical signals generated by any subset of the subpixels 150S. The switch 160 may comprise an accumulator 309 electrically connected to the discrete portions of the electrical contact 119B associated with the subpixels 150S, for example, through the sub-switches 311. The accumulator 309 is configured to combine the electrical signals generated by any subset of the subpixels 150S. In an embodiment, the accumulator 309 is configured to collect charge carriers from the subpixels 150S. In an embodiment, the accumulator 309 includes a capacitor 308 in the feedback path of an op-amp 312. Charge carriers from the subpixels 150S accumulate on the capacitor 308 over a period of time (“integration period”). After the integration period has expired, the voltage across the capacitor 308 is sampled and then reset by a reset switch 305. When a subpixel 150S is excluded from the subset, the charge carriers therefrom may be prevented from reaching the accumulator 309.

The electronics layer 120 of the radiation detector 100 may include an electronic system 121 suitable for processing or interpreting signals generated by the pixels 150 from the radiation incident thereon. The electronic system 121 is electrically connected to the discrete portions of the electric contact 119B of a pixel 150, for example, via the switch 160. The electronic system 121 may include analog circuitry such as a filter network, amplifiers, integrators, and comparators, or a digital circuitry such as a microprocessor, and memory. The electronic system 121 may include one or more ADCs. The electronic system 121 may include components shared by multiple pixels 150 or components dedicated to a single pixel 150. The electronic system 121 may include components shared by all of the subpixels 150S of a pixel 150 or components dedicated to a single subpixel 150S. For example, the electronic system 121 may include an amplifier that is dedicated to a pixel 150 and shared among all the subpixels 150S of this pixel 150, and a microprocessor that is shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using vias.

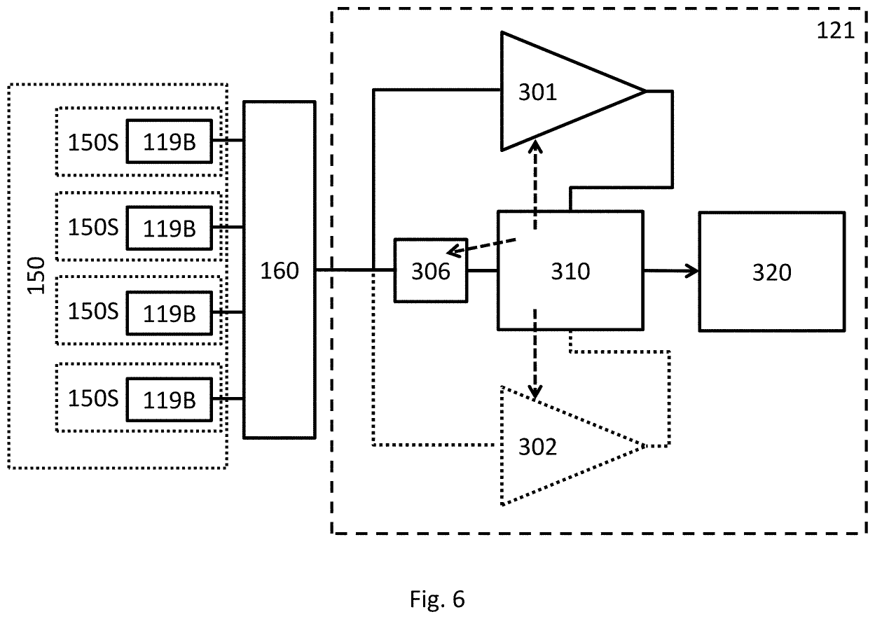

FIG. 6 shows a component diagram of the electronic system 121, according to an embodiment. In this embodiment, the electronic system 121 includes a comparator 301, a counter 320, a meter 306 and a controller 310.

The comparator 301 is configured to compare an output signal from the switch 160, which represents the combined electrical signals generated by the subset of the subpixels 150S, to an output threshold. The comparator 301 may be controllably activated or deactivated by the controller 310. The comparator 301 may be a continuous comparator. Namely, the comparator 301 may be configured to be activated continuously and monitor the output signal continuously. The first comparator 301 may be a clocked comparator. The output threshold may be 5-10%, 10%-20%, 20-30%, 30-40% or 40-50% of the output signal a single particle of radiation may generate on the switch 160.

The comparator 301 may include one or more op-amps or any other suitable circuitry. The comparator 301 may have a high speed to allow the system 121 to operate under a high flux of incident radiation.

The counter 320 is configured to register a number of particles of radiation reaching a pixel 150. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the comparator 301 determines that the absolute value of the output signal equals or exceeds the absolute value of the output threshold (e.g., the absolute value of the output signal increases from below the absolute value of the output threshold to a value equal to or above the absolute value of the output threshold). The absolute value is used here because the output signal may be negative or positive. The controller 310 may be configured to keep deactivated the counter 320 and any other circuits the operation of the comparator 301 does not require, before the time at which the comparator 301 determines that the absolute value of the output signal equals or exceeds the absolute value of the output threshold. The time delay may expire before or after the output signal becomes stable, i.e., the rate of change of the output signal is substantially zero. The phase “the rate of change of the output signal is substantially zero” means that temporal change of the output signal is less than 0.1%/ns. The phase “the rate of change of the output signal is substantially non-zero” means that temporal change of the output signal is at least 0.1%/ns.

The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the comparator 301 activates the controller 310 when the absolute value of the output signal equals or exceeds the absolute value of the output threshold.

The controller 310 may be configured to cause the meter 306 to measure the output signal upon expiration of the time delay. The controller 310 may be configured to connect the discrete portions of the electric contact 119B to an electrical ground, so as to discharge any charge carriers accumulated thereon. The controller 310 may connect the discrete portions of the electric contact 119B to the electrical ground by controlling the switch 305. The switch may be a transistor such as a field-effect transistor (FET).

In an embodiment, the electronic system 121 has no analog filter network (e.g., a RC network). In an embodiment, the electronic system 121 has no analog circuitry.

The meter 306 may feed the output signal it measures to the controller 310 as an analog or digital signal.

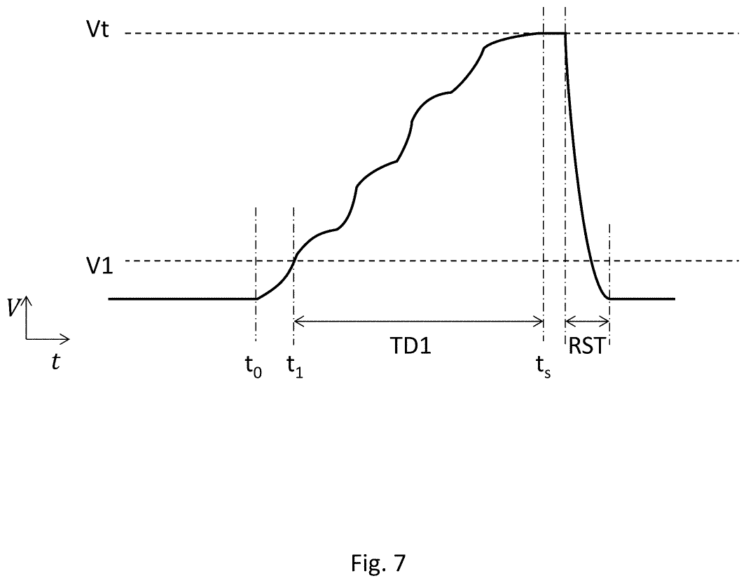

FIG. 7 schematically shows a temporal change of the output signal, caused by charge carriers generated by one or more particles of radiation incident on a pixel 150, according to an embodiment. When one or more particles of radiation hit the pixel 150 starting at time to, the absolute value of the output signal starts to increase. At time t₁, the comparator 301 determines that the absolute value of the output signal equals or exceeds the absolute value of the output threshold V1, and the controller 310 starts the time delay TD1 and the controller 310 may deactivate the comparator 301 at the beginning of TD1. If the controller 310 is deactivated before t₁, the controller 310 is activated at t₁. At time tₛ, the time delay TD1 expires. The particles of radiation may continue hit the pixel 150 throughout the entirety of TD1.

The controller 310 may be configured to cause the meter 306 to measure the output signal upon expiration of the time delay TD1. The output signal Vt measured by the meter 306 is proportional to the amount of charge carriers generated by the incident particles of radiation on the pixel 150 from t₀ to tₛ, which relates to the total energy of the incident particles of radiation. When the incident particles of radiation have similar energy, the controller 310 may be configured to determine the number of incident particles of radiation from t₀ to tₛ, by dividing Vt with the output signal that a single particle of radiation would cause on the switch 160. The controller 310 may increase the counter 320 by the number of particles of radiation.

After TD1 expires, the controller 310 connects the discrete portions of the electric contact 119B to an electric ground for a reset period RST to allow charge carriers accumulated thereon to flow to the ground. After RST, the electronic system 121 is ready to detect another incident particle of radiation. If the comparator 301 has been deactivated, the controller 310 can activate it at any time before RST expires. If the controller 310 has been deactivated, it may be activated before RST expires.

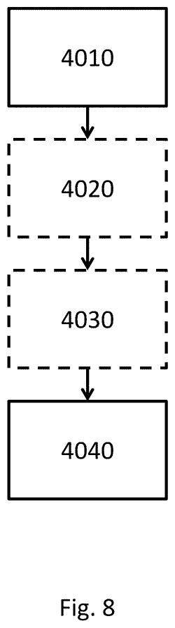

FIG. 8 shows a flow chart for a method suitable for detecting radiation using the radiation detector 100. In procedure 4010, a subset of the plurality of subpixels 150S in a pixel 150 is identified. In optional procedure 4020, a magnitude of the electrical signal generated by each of the subpixels 150S in the subset is determined, for example, using the sub-switch 311 connected thereto. In optional procedure 4030, that subpixel 150S is disconnected, for example, by the sub-switch 311 connected thereto, i.e. disconnecting the subpixel using the sub-switch connected thereto upon determination that the magnitude of the electrical signal generated by that subpixel 150S equals to or exceeds a magnitude threshold. In procedure 4040, the electrical signals generated by the subset of the subpixels 150S are combined. In an embodiment, the subset includes all of the non-defective subpixels 150S of a pixel 150 and none of the defective subpixels 150S in the pixel 150.

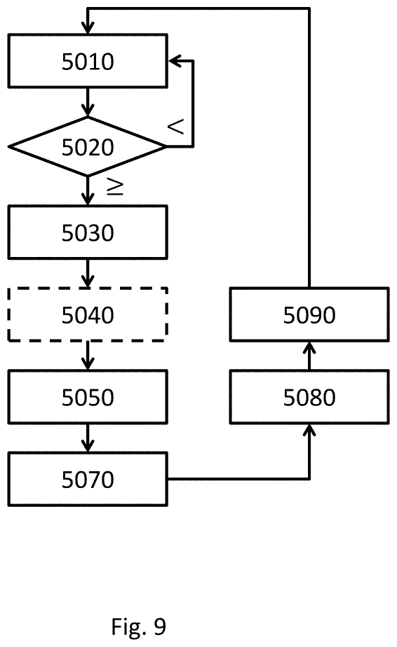

FIG. 9 shows a flow chart for a method suitable for detecting radiation incident on a pixel 150 using a system such as the system 121 operating as shown in FIG. 6. In procedure 5010, the output signal of the switch 160 is compared to the output threshold, e.g., using the comparator 301. In procedure 5020, whether the absolute value of the output signal equals or exceeds the absolute value of the output threshold V1 is determined, e.g., with the controller 310. If the absolute value of the output signal does not equal or exceed the absolute value of the output threshold, the method goes back to procedure 5010. If the absolute value of the output signal equals or exceeds the absolute value of the output threshold, continue to procedure 5030. In procedure 5030, the time delay TD1 is started, e.g., using the controller 310. In optional procedure 5040, a circuit (e.g., the counter 320) is activated, e.g., using the controller 310, during the time delay TD1 (e.g., at the expiration of TD1). In procedure 5050, the output signal is measured, e.g., using the meter 306, upon expiration of the time delay TD1. In procedure 5070, the number of particles of radiation incident on the pixel 150 from t₀ to tₛ is determined by dividing the output signal measured by an output signal a single particle of radiation would cause on the switch 160. The output signal that a single particle of radiation would cause on the switch 160 may be known or measured separately in advance. In procedure 5080, the counter is increased by the number of particles of radiation. The method goes to procedure 5090 after procedure 5080. In procedure 5090, reset the output signal, e.g., by connecting the discrete portions of the electric contact 119B in the pixel 150 to an electrical ground.

According to an embodiment, the detector 100 may use delta-sigma (sigma-delta, ΔΣ or ΣΔ) modulation. In a conventional ADC, an analog signal is integrated, or sampled, with a sampling frequency and subsequently quantized in a multi-level quantizer into a digital signal. This process introduces quantization error noise. The first step in a delta-sigma modulation is delta modulation. In delta modulation the change in the signal (its delta) is encoded, rather than the absolute value. The result is a stream of pulses, as opposed to a stream of numbers. The digital output (i.e., the pulses) is passed through a 1-bit DAC and the resulting analog signal (sigma) is added to the input signal of the ADC. During the integration of the analog signal, when the analog signal reaches the delta, a counter is increased by one and the delta is deducted from the analog signal. At the end of the integration, the registered value of the counter is the digital signal and the remaining analog signal smaller than the delta is the residue analog signal.

The electronic system 121 may further include another comparator 302 but omit the meter 306, as shown in FIG. 6. During TD1, whenever the comparator 302 determines that the output signal reaches Vp, which is the output signal a single incident particle of radiation would have caused on the switch 160, the controller 310 connects the discrete portions of the electric contact 119B in the pixel 150 to an electric ground to allow charge carriers accumulated thereon to flow to the ground and increases the counter 320 by one.

After TD1 expires, the controller 310 again connects the discrete portions of the electric contact 119B in the pixel 150 to an electric ground for a reset period RST to allow charge carriers accumulated thereon to flow to the ground. The number of the counter 320 at the expiration of TD1 represents the number of incident particles of radiation on the pixel 150 from t₀ to the expiration of TD1.

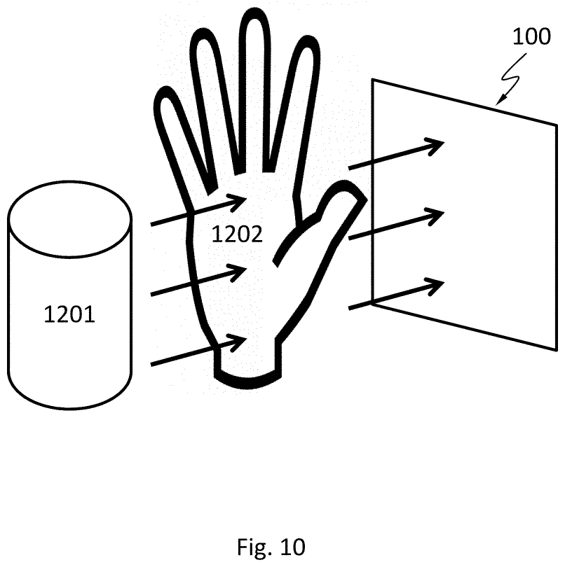

FIG. 10 schematically shows a system comprising the radiation detector 100 described herein. The system may be used for medical imaging such as chest X-ray radiography, abdominal X-ray radiography, etc. The system comprises an X-ray source 1201. X-ray emitted from the X-ray source 1201 penetrates an object 1202 (e.g., a human body part such as chest, limb, abdomen), is attenuated by different degrees by the internal structures of the object 1202 (e.g., bones, muscle, fat and organs, etc.), and is projected to the radiation detector 100. The radiation detector 100 forms an image by detecting the intensity distribution of the X-ray.

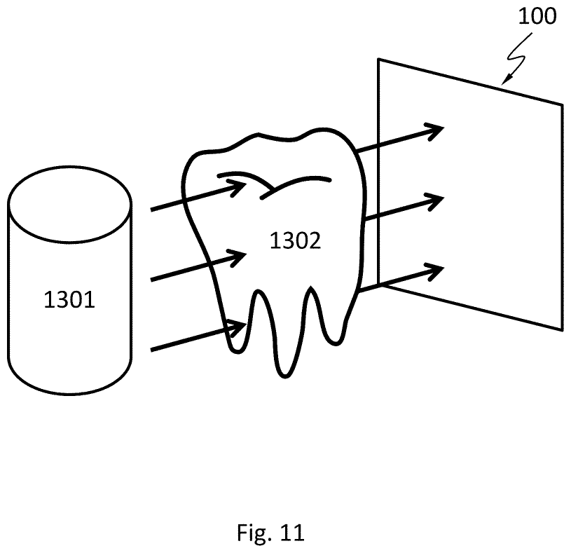

FIG. 11 schematically shows a system comprising the radiation detector 100 described herein. The system may be used for medical imaging such as dental X-ray radiography. The system comprises an X-ray source 1301. X-ray emitted from the X-ray source 1301 penetrates an object 1302 that is part of a mammal (e.g., human) mouth. The object 1302 may include a maxilla bone, a palate bone, a tooth, the mandible, or the tongue. The X-ray is attenuated by different degrees by the different structures of the object 1302 and is projected to the radiation detector 100. The radiation detector 100 forms an image by detecting the intensity distribution of the X-ray. Teeth absorb X-ray more than dental caries, infections, periodontal ligament. The dosage of X-ray radiation received by a dental patient is typically small (around 0.150 mSv for a full mouth series).

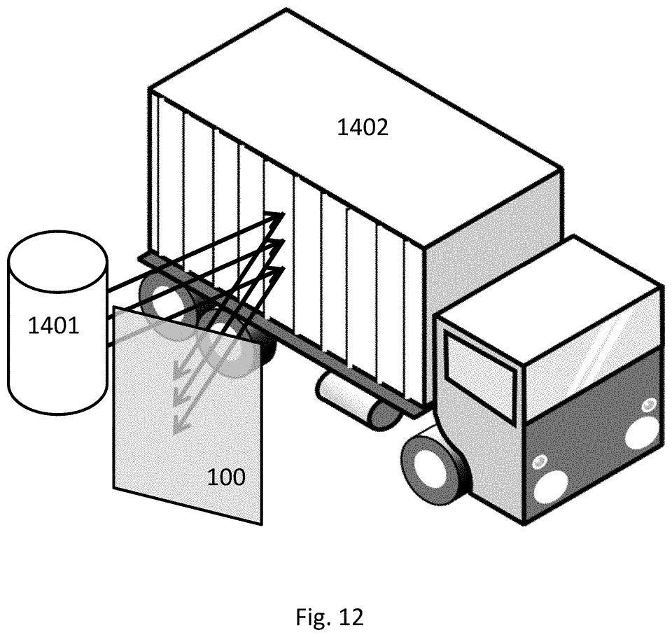

FIG. 12 schematically shows a cargo scanning or non-intrusive inspection (NII) system comprising the radiation detector 100 described herein. The system may be used for inspecting and identifying goods in transportation systems such as shipping containers, vehicles, ships, luggage, etc. The system comprises an X-ray source 1401. X-ray emitted from the X-ray source 1401 may backscatter from an object 1402 (e.g., shipping containers, vehicles, ships, etc.) and be projected to the radiation detector 100. Different internal structures of the object 1402 may backscatter X-ray differently. The radiation detector 100 forms an image by detecting the intensity distribution of the backscattered X-ray and/or energies of the backscattered X-ray particles of radiation.

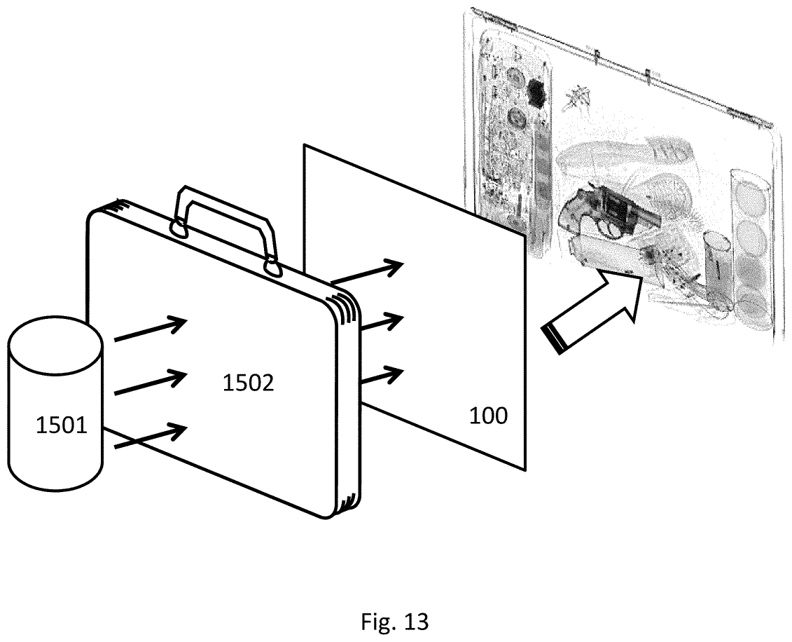

FIG. 13 schematically shows another cargo scanning or non-intrusive inspection (NII) system comprising the radiation detector 100 described herein. The system may be used for luggage screening at public transportation stations and airports. The system comprises an X-ray source 1501. X-ray emitted from the X-ray source 1501 may penetrate a piece of luggage 1502, be differently attenuated by the contents of the luggage, and projected to the radiation detector 100. The radiation detector 100 forms an image by detecting the intensity distribution of the transmitted X-ray. The system may reveal contents of luggage and identify items forbidden on public transportation, such as firearms, narcotics, edged weapons, flammables.

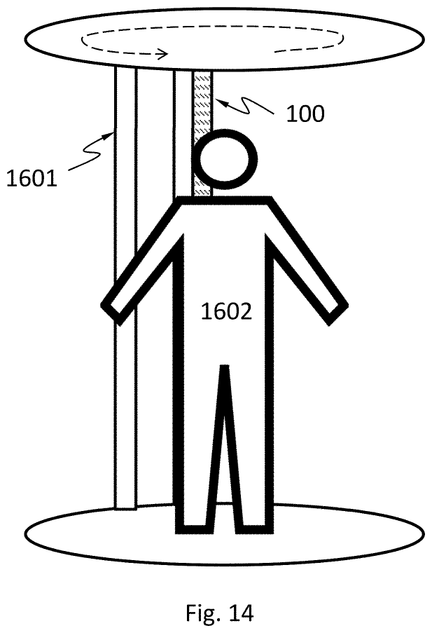

FIG. 14 schematically shows a full-body scanner system comprising the radiation detector 100 described herein. The full-body scanner system may detect objects on a person's body for security screening purposes, without physically removing clothes or making physical contact. The full-body scanner system may be able to detect non-metal objects. The full-body scanner system comprises an X-ray source 1601. X-ray emitted from the X-ray source 1601 may backscatter from a human 1602 being screened and objects thereon, and be projected to the radiation detector 100. The objects and the human body may backscatter X-ray differently. The radiation detector 100 forms an image by detecting the intensity distribution of the backscattered X-ray. The radiation detector 100 and the X-ray source 1601 may be configured to scan the human in a linear or rotational direction.

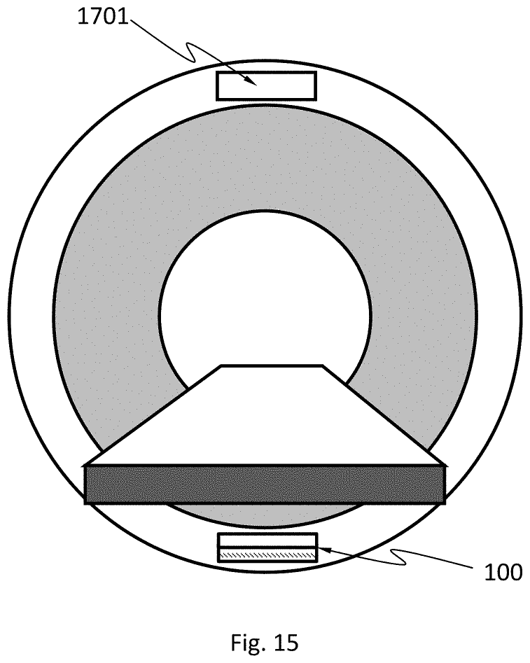

FIG. 15 schematically shows an X-ray computed tomography (X-ray CT) system. The X-ray CT system uses computer-processed X-rays to produce tomographic images (virtual “slices”) of specific areas of a scanned object. The tomographic images may be used for diagnostic and therapeutic purposes in various medical disciplines, or for flaw detection, failure analysis, metrology, assembly analysis and reverse engineering. The X-ray CT system comprises the radiation detector 100 described herein and an X-ray source 1701. The radiation detector 100 and the X-ray source 1701 may be configured to rotate synchronously along one or more circular or spiral paths.

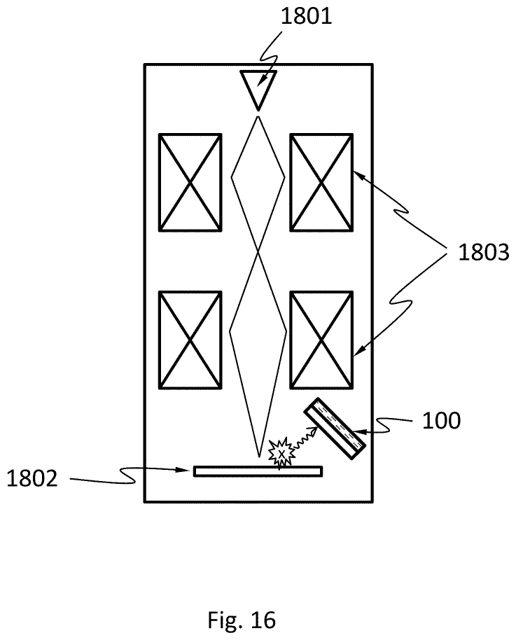

FIG. 16 schematically shows an electron microscope. The electron microscope comprises an electron source 1801 (also called an electron gun) that is configured to emit electrons. The electron source 1801 may have various emission mechanisms such as thermionic, photocathode, cold emission, or plasmas source. The emitted electrons pass through an electronic optical system 1803, which may be configured to shape, accelerate, or focus the electrons. The electrons then reach a sample 1802 and an image detector may form an image therefrom. The electron microscope may comprise the radiation detector 100 described herein, for performing energy-dispersive X-ray spectroscopy (EDS). EDS is an analytical technique used for the elemental analysis or chemical characterization of a sample. When the electrons incident on a sample, they cause emission of characteristic X-rays from the sample. The incident electrons may excite an electron in an inner shell of an atom in the sample, ejecting it from the shell while creating an electron hole where the electron was. An electron from an outer, higher-energy shell then fills the hole, and the difference in energy between the higher-energy shell and the lower energy shell may be released in the form of an X-ray. The number and energy of the X-rays emitted from the sample can be measured by the radiation detector 100.

The radiation detector 100 described here may have other applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A radiation detector, comprising:
a pixel comprising a plurality of subpixels, each of the subpixels configured to generate an electrical signal upon exposure to a radiation;
a switch electrically connected to the plurality of subpixels;
wherein the switch is configured to combine electrical signals generated by a subset of the subpixels.

2. The radiation detector according to claim 1, wherein the switch is configured to detect a magnitude of the electrical signal generated by each of the subpixels.

3. The radiation detector according to claim 2, wherein the switch is configured to disconnect any one of the subpixels when the magnitude of the electrical signal generated by that subpixel exceeds a magnitude threshold.

4. The radiation detector according to claim 1, wherein the switch comprises a plurality of sub-switches respectively connected to the subpixels.

5. The radiation detector according to claim 4, wherein each of the sub-switches is configured to detect a magnitude of the electrical signal generated by the subpixel connected thereto.

6. The radiation detector according to claim 5, wherein each of the sub-switches is configured to disconnect the subpixel connected thereto when the magnitude exceeds a magnitude threshold.

7. The radiation detector according to claim 1, wherein the pixel comprises four subpixels.

8. The radiation detector according to claim 1, wherein the pixel further comprises a radiation absorption layer.

9. The radiation detector according to claim 8, wherein the radiation absorption layer comprises a semiconductor.

10. The radiation detector according to claim 9, wherein the semiconductor is selected from a group consisting of silicon, germanium, GaAs, CdTe, CdZnTe, and combinations thereof.

11. The radiation detector according to claim 1, wherein the switch further comprises an accumulator to combine the electrical signals generated by any subset of the subpixels.

12. The radiation detector according to claim 1, further comprising:
a comparator configured to compare an output signal from the switch to an output threshold;
a counter configured to register a number of particles of radiation absorbed by radiation detector;
a controller;
a meter configured to measure the output signal;
wherein the controller is configured to start a time delay from a time at which the comparator determines that an absolute value of the output signal equals or exceeds an absolute value of the output threshold;
wherein the controller is configured to cause the meter to measure the output signal upon expiration of the time delay;
wherein the controller is configured to determine a number of particles by dividing the output signal measured by the meter by an output signal of the switch that a single particle generates;
wherein the controller is configured to cause the number registered by the counter to increase by the number of particles.

13. The radiation detector of claim 12, wherein the controller is configured to deactivate the comparator at a beginning of the time delay.

14. The radiation detector of claim 12, wherein the output threshold is 5-10% of the output signal of the switch that a single particle generates.

15. A system comprising the radiation detector of claim 1 and an X-ray source, wherein the system is configured to perform X-ray radiography on human chest or abdomen.

16. A system comprising the radiation detector of claim 1 and an X-ray source, wherein the system is configured to perform X-ray radiography on human mouth.

17. A cargo scanning or non-intrusive inspection (NII) system, comprising the radiation detector of claim 1 and an X-ray source, wherein the cargo scanning or non-intrusive inspection (NII) system is configured to form an image using backscattered X-ray.

18. A cargo scanning or non-intrusive inspection (NII) system, comprising the radiation detector of claim 1 and an X-ray source, wherein the cargo scanning or non-intrusive inspection (NII) system is configured to form an image using X-ray transmitted through an object inspected.

19. A full-body scanner system comprising the radiation detector of claim 1 and an X-ray source.

20. An X-ray computed tomography (X-ray CT) system comprising the radiation detector of claim 1 and an X-ray source.

21. An electron microscope comprising the radiation detector of claim 1, an electron source and an electronic optical system.

22. A system comprising the radiation detector of claim 1, wherein the system is an X-ray telescope, or an X-ray microscopy, or wherein the system is configured to perform mammography, industrial defect detection, microradiography, casting inspection, weld inspection, or digital subtraction angiography.

23. A method comprising:
obtaining a radiation detector comprising a pixel, wherein the pixel comprises a plurality of subpixels, each of the subpixels being configured to generate an electrical signal upon exposure to a radiation;
identifying a subset of the subpixels;
combining the electrical signals generated by the subset of the subpixels.

24. The method according to claim 23, wherein the radiation detector comprises a switch electrically connected to the plurality of subpixels, and the switch comprises a plurality of sub-switches respectively connected to the subpixels.

25. The method according to claim 24, further comprising detecting a magnitude of the electrical signal generated by each subpixel using the sub-switch connected thereto.

26. The method according to claim 25, further comprising disconnecting that subpixel using the sub-switch connected thereto upon determination that the magnitude exceeds a magnitude threshold.
